(19) United States
(12) Patent Application Publication
(10) Pub. No.: US 2026/0325350 A1
(43) Pub. Date: Oct. 22, 2026

(54) Systems and Methods for Controlling Continuous Irrigation in Surgical Systems

(71) Applicant: Johnson & Johnson Surgical Vision, Inc., Irvine, CA (US)

(72) Inventors: Deep Mehta, Irvine, CA (US); Edith Fung, Diamond Bar, CA (US); Jacqueline Villalobos, Chula Vista, CA (US); Linyi Xia, Irvine, CA (US); Hajime Minoguchi, Irvine, CA (US)

(21) Appl. No.: 19/724,615

(22) Filed: Jun. 29, 2026

Related U.S. Application Data
(63) Continuation-in-part of application No. 17/064,178, filed on Oct. 6, 2020, now Pat. No. 12,678,326.
(60) Provisional application No. 62/913,669, filed on Oct. 10, 2019.

(51) Int. Cl. A61F 9/007

(52) U.S. Cl. CPC A61F 9/00745

(57) ABSTRACT

Systems and methods for controlling continuous irrigation in surgical systems are disclosed. Continuous irrigation is automatically disabled when the foot pedal position is at a zero position and a determined pressure at the tip of a handpiece falls within a predetermined pressure range associated with a determined operating mode for a predefined time period. The system determines a pressure at the tip of the handpiece, determines an operating mode of the system, the operating mode being one of a phacoemulsification mode, vitrectomy mode, and an irrigation/aspiration mode, and compares the determined pressure at the tip of the handpiece to a predetermined pressure range associated with the determined operating mode.

### CROSS-REFERENCE TO RELATED APPLICATIONS

This application is a continuation-in-part of U.S. Patent Application No. 17/064,178 filed Oct. 6, 2020, which claims the benefit under 35 U.S.C. § 119(e) of U.S. Provisional Patent Application No. 62/913,669 filed Oct. 10, 2019, each of which are incorporated herein by reference in their entirety.

### FIELD OF THE INVENTION

The present invention relates to phacoemulsification fluidics system control, and, more particularly to systems and methods for controlling continuous irrigation in surgical systems.

### BACKGROUND

Cataracts affect more than 22 million Americans age 40 and older. And as the U.S. population ages, more than 30 million Americans are expected to have cataracts by the year 2020. Cataract surgery entails the removal of a lens of an eye that has developed clouding of the eye’s natural lens, or opacification. As a result of opacification, light is unable to travel to the retina, thereby causing vision loss. Once vision becomes impaired, cataract surgery is a viable option with a high level of success. During cataract surgery, a surgeon replaces the clouded lens with an intraocular lens (IOL).

Certain surgical procedures, such as phacoemulsification surgery, have been successfully employed in the treatment of certain ocular problems, such as cataracts. Phacoemulsification surgery utilizes a small corneal incision to insert the tip of at least one phacoemulsification handheld surgical implement, or handpiece, through the corneal incision. The handpiece includes a needle which is ultrasonically driven once placed within the incision to emulsify the eye lens, or to break the cataract into small pieces. The broken cataract pieces or emulsified eye lens may subsequently be removed using the same handpiece, or another handpiece, in a controlled manner. The surgeon may then insert an IOL into the eye through the incision. The incision is allowed to heal, and the result for the patient is typically significantly improved eyesight.

During the phacoemulsification process for cataract removal, a single plastic cassette (which may be disposable or reusable) is generally used to collect effluent material. This single cassette requires a prime on every insertion. The cost per case is a very sensitive factor for surgeons, which includes balanced salt solution (“BSS”) usage and cassette cost. Currently a significant portion of BSS is used during prime to fill the line from the BSS bottle or bag to the pack. Time and coordination is also required for a non-sterile nurse to spike and hang the BSS bottle after the sterile nurse has inserted the cassette. This can slow down the setup procedure as the sterile nurse may be waiting for this to occur to start prime. The irrigation side of the cassette has maintained sterility, but since it is physically part of the cassette, it is typically disposed of at the end of a case, in some cases along with the BSS bottle.

Current phacoemulsification platforms provide multiple methods to activate irrigation during surgery, e.g., a graphical user interface (GUI) button, remote control, and a foot pedal control to toggle irrigation. Many systems provide a continuous irrigation button on the graphical user interface which toggles the irrigation on and off. When continuous irrigation is on, the system actuates the irrigation valve to let the irrigation fluid from the source to reach the distal end of the surgical hand piece via a sleeve. A surgeon can also choose to activate irrigation with foot pedal control. In this case, a foot pedal treadle travel is divided into three segments: irrigation, irrigation/aspiration and irrigation/aspiration/ultrasound power. When a surgeon pushes the foot pedal treadle to the first position of the travel, the system actuates the irrigation valve to an on position. On the other hand, when the surgeon moves the treadle back up to the initial position (foot pedal position zero (FP0)), the system actuates the irrigation valve to the off position. In addition, a foot pedal switch can be programmed to turn on or turn off continuous irrigation or use the foot pedal tapping feature to accomplish the same result. Having a foot pedal control often helps the surgeon in turning the irrigation off when traversing between phacoemulsification (phaco), irrigation and aspiration (IA) and vitrectomy (Vit) modes of the surgery, but does so at the physical control of the operator and may be latent in the desired or optimal time for such functionality to occur. Similarly, use of a continuous irrigation button on a GUI does not allow a surgeon to control the irrigation when traversing between surgical modes except pressing the GUI button prior to switching the surgical mode. Thus, the need exists for the automated control of continuous irrigation in a surgical system.

### BRIEF DESCRIPTION OF THE DRAWINGS

This disclosure is illustrated by way of example and not by way of limitation in the accompanying figure(s). The figure(s) may, alone or in combination, illustrate one or more embodiments of the disclosure. Elements illustrated in the figure(s) are not necessarily drawn to scale. Reference labels may be repeated among the figures to indicate corresponding or analogous elements.

The detailed description makes reference to the accompanying figures in which:

FIG. 1A is a schematic illustrating an eye treatment system in which a cassette is coupled to an eye treatment probe with an eye treatment console under one example;

FIG. 1B is a schematic illustrating an ophthalmic surgical treatment console under another exemplary example;

FIG. 2 is a flow diagram illustrating an example of the disclosed invention;

FIGS. 3A and 3B illustrate system conditions accordance with another example of the disclosed invention;

FIG. 4. illustrate system conditions accordance with an alternate example of the disclosed invention;

FIGS. 5A and 5B are collectively a flow diagram illustrating an example of the present invention;

FIG. 6 is a flow diagram illustrating another example of the disclosed invention; and

FIG. 7 is a graph illustrating a pressure differential according with an example of the disclosed invention.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Examples are provided throughout so that this disclosure is sufficiently thorough and fully conveys the scope of the disclosed examples to those who are skilled in the art. Numerous specific details are set forth, such as examples of specific components, devices, and methods, to provide a thorough understanding of examples of the present disclosure. Nevertheless, it will be apparent to those skilled in the art that certain specific disclosed details need not be employed, and that examples may be embodied in different forms. As such, the examples should not be construed to limit the scope of the disclosure. As referenced above, in some examples, well-known processes, well-known device structures, and well-known technologies may not be described in detail.

The disclosed allows for the irrigation functionality of a phacoemulsification system to be decoupled from the aspiration, thus allowing for extended use of an irrigation cassette across multiple patients, minimizing cost per case, as well as setup time for the surgical center.

Phacoemulsification platforms may provide two main methods to activate irrigation during surgery: a graphical user interface (GUI) button and/or a foot pedal control to toggle irrigation. Phacoemulsification systems which provide a continuous irrigation function usually do so through a button actuation on a GUI which may toggle the irrigation on and off. When continuous irrigation is on, the system actuates the irrigation valve to let the irrigation fluid from the source reach the distal end of the surgical hand piece via a sleeve. When continuous irrigation is turned off, the system actuates the irrigation valve to an off position to stop the irrigation fluid from reaching the distal end of the hand piece.

A surgeon may also choose to activate the irrigation with foot pedal control. In such a case, a foot pedal treadle travel may be divided into three segments: irrigation, irrigation/aspiration and irrigation/aspiration/ultrasound power. For example, when a surgeon pushes the foot pedal treadle to a first position of travel, the system may actuate the irrigation valve to an on position. Similarly, when a surgeon moves the treadle back up to an initial position or foot pedal idle position, for example, the system may actuate the irrigation valve to an off position. Having a foot pedal control often helps the surgeon in turning the irrigation off when traversing between phaco, IA or vitrectomy modes of the surgery whereas with continuous irrigation button on the GUI, the surgeon is not able to control the irrigation when traversing between surgical modes except by (1) pressing the GUI button prior to switching the surgical mode; (2) programming a switch or button on a foot pedal to turn on or turn off continuous irrigation; or (3) use a foot pedal tapping feature to accomplish the same result.

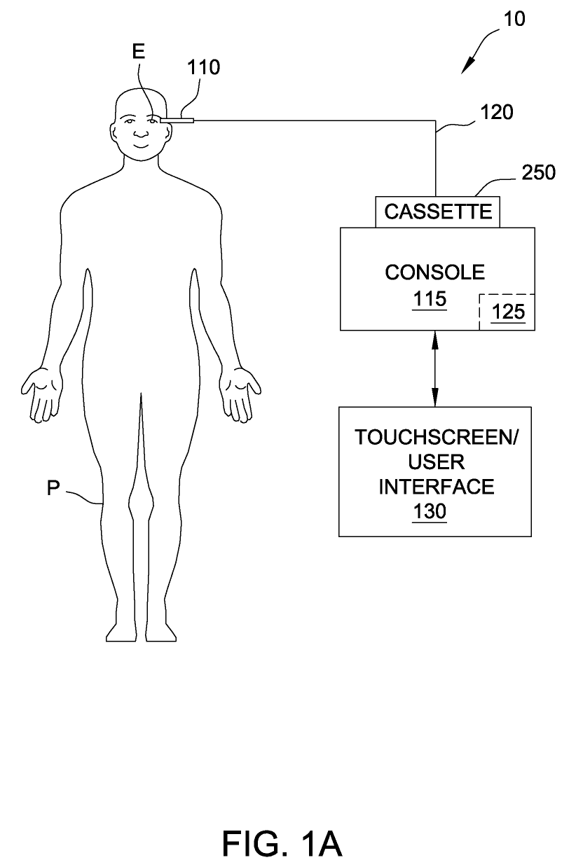

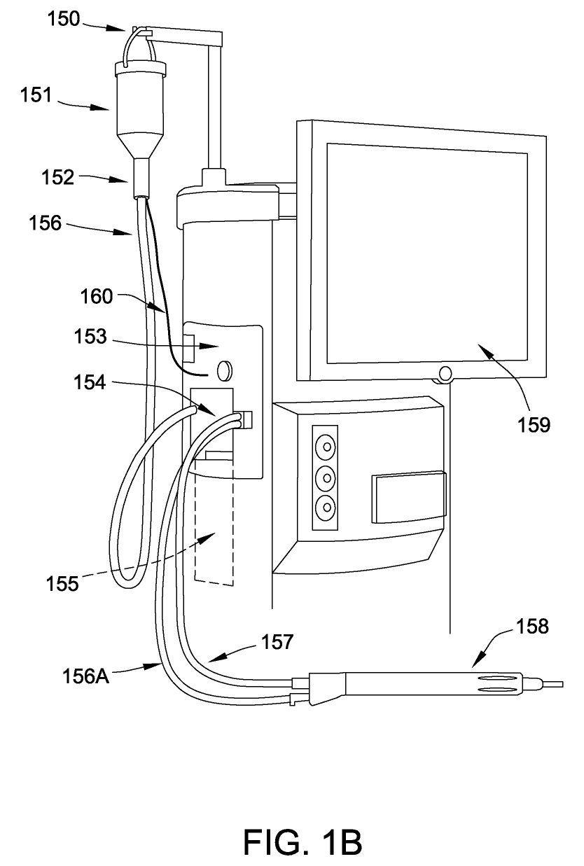

Referring now to FIG. 1A, a system 10 for treating an eye E of a patient P generally includes an eye treatment probe handpiece 110 coupled with a console 115 by a cassette 250. Handpiece 110 generally includes a handle for manually manipulating and supporting an insertable probe tip. The probe tip has a distal end which is insertable into the eye, with one or more lumens in the probe tip allowing irrigation fluid to flow from console 115 and/or cassette 250 into the eye. Aspiration fluid may also be withdrawn through a lumen of the probe tip, with console 115 and cassette 250 generally including a vacuum aspiration source, a positive displacement aspiration pump, or both to help withdraw and control a flow of surgical fluids into and out of eye E. As the surgical fluids may include biological materials that should not be transferred between patients, cassette 250 will often comprise a sterilizable (or alternatively, disposable) structure, with the surgical fluids being transmitted through flexible conduits 120 of cassette 250 that avoid direct contact in between those fluids and the components of console 115.

When a distal end of the probe tip of handpiece 110 is inserted into an eye E, for example, for removal of a lens of a patient P with cataracts, an electrical conductor and/or pneumatic line (not shown) may supply energy from console 115 to an ultrasound transmitter of handpiece 110, a cutter mechanism, or the like. Alternatively, handpiece 110 may be configured as an IA and/or vitrectomy handpiece. Also, the ultrasonic transmitter may be replaced by other means for emulsifying a lens, such as a high energy laser beam. The ultrasound energy from handpiece 110 helps to fragment the tissue of the lens, which can then be drawn into a port of the tip by aspiration flow. To balance the volume of material removed by the aspiration flow, an irrigation flow through handpiece 110 (or a separate probe structure) may also be provided, with both the aspiration and irrigation flows being controlled by console 115.

To avoid cross-contamination between patients without incurring excessive expenditures for each procedure, cassette 250 and its flexible conduits 120 may be disposable. However, the flexible conduit or tubing may be disposable, with the cassette body and/or other structures of the cassette being sterilizable. Cassette 250 may be configured to interface with reusable components of console 115, including, but not limited to, peristaltic pump rollers, a Venturi or other vacuum source, a controller 125, and/or the like.

Console 115 may include controller 125, which may include an embedded microcontroller and/or many of the components common to a personal computer, such as a processor, data bus, a memory, input and/or output devices (including a user interface 130 (e.g. touch screen, graphical user interface (GUI), etc.), and the like. Controller 125 will often include both hardware and software, with the software typically comprising machine readable code or programming instructions for implementing one, some, or all of the methods described herein. The code may be embodied by a tangible media such as a memory, a magnetic recording media, an optical recording media, or the like. Controller 125 may have (or be coupled with) a recording media reader, or the code may be transmitted to controller 125 by a network connection such as an internet, an intranet, an ethernet, a wireless network, or the like. Along with programming code, controller 125 may include stored data for implementing the methods described herein and may generate and/or store data that records parameters corresponding to the treatment of one or more patients.

Referring now to FIG. 1B, a simplified surgical console is illustrated, where a fluid path may be demonstrated under an example. In this example, an irrigation source 151 may be configured as a bottle or bag hanging from an IV pole hanger 150. It is understood by those skilled in the art that, while an integrated IV pole is illustrated, other configurations, utilizing standalone/static IV poles, or other suitable configurations, are contemplated by the present disclosure. In addition, the irrigation source may be located within the system console.

An exemplary irrigation path for fluid may be realized via tubing cassette 154 having cassette tubing interface 153, which receives fluid from irrigation source 151 via drip chamber 152. Irrigation line 156A and aspiration line 157 are coupled to handpiece 158. Irrigation fluid may flow from drip chamber 152 through the irrigation tubing 156 into tubing cassette 154. Irrigation fluid may then flow from the tubing cassette 154 through handpiece irrigation line 156A which may be coupled to an irrigation port on handpiece 158. Aspirated fluid may flow from handpiece aspiration line 157 back to tubing cassette 154 and into a waste collection bag 155. A touch screen display 159 may be provided to display system operation conditions and parameters, and may include a user interface (e.g., touch screen, keyboard, track ball, mouse, etc. – see controller 125 of FIG. 1A) for entering data and/or instructions to the system of FIG. 1B.

The present invention provides a system and method for turning off continuous irrigation within the surgical system when a surgeon removes the hand piece out of the eye (i.e. anterior chamber) and, for example, turns the continuous irrigation back on when hand piece is reinserted to proceed with the next step of the surgery. Typically, once substantially all the cataract particles are emulsified and evacuated out of the chamber using a phaco hand piece, the surgeon may remove the hand piece out of the chamber, disconnects the irrigation and aspiration luers from the phaco hand piece, and connects them to the IA hand piece. The IA hand piece may then be inserted into the chamber using the same incision. When performing vitrectomy, the aspiration luer may be connected to the vitrectomy hand piece and the irrigation luer may be either connected to the vitrectomy hand piece or to a trocar instrument with a separate incision. The present invention may turn the continuous irrigation off when certain trigger events are detected.

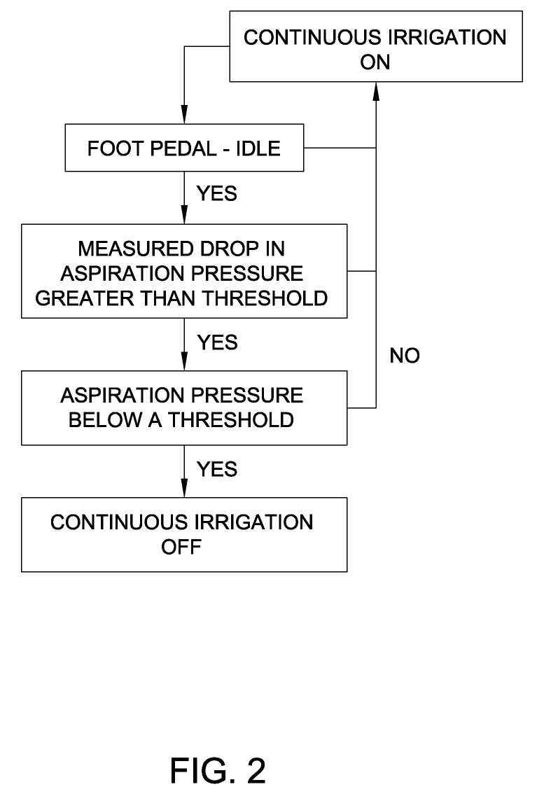

As illustrated in FIG. 2, such trigger events may include, but are not limited to the following conditions:
1. Continuous irrigation is on and the feature is active, AND
2. Foot pedal treadle is at Idle (FP 0) position, AND
3. Aspiration pressure is decreasing at a rate less than a certain specified value or dropped to a specified limit.

In an example of the present invention, the system may turn the continuous irrigation off when one or more trigger events are detected during, for example, phaco, IA, or Vit modes. By way of non-limiting example, the present invention may turn on continuous irrigation if one or more of the following trigger events are detected in the phaco/IA/Vit mode:
1. Continuous irrigation was turned off manually or automatically, AND
2. Foot pedal treadle is at irrigation (foot pedal 1 (FP1)) or irrigation/aspiration (FP2) or irrigation/aspiration/ultrasound power (FP3) position.

In an alternative example, a user of the present invention, such as a surgeon, for example, may turn the continuous irrigation on manually by pressing a GUI presented switch prior to reentering the chamber. The proposed auto turn-off feature may be enabled once the continuous irrigation button is pressed or selected on the GUI or a switch/button on the foot pedal is activated. The auto turn off feature may be disabled when the continuous irrigation button is deselected on the GUI or the switch/button on the foot pedal is activated.

In an example, continuous irrigation may be turned off when a user changes from one mode to another, e.g. phaco to IA.

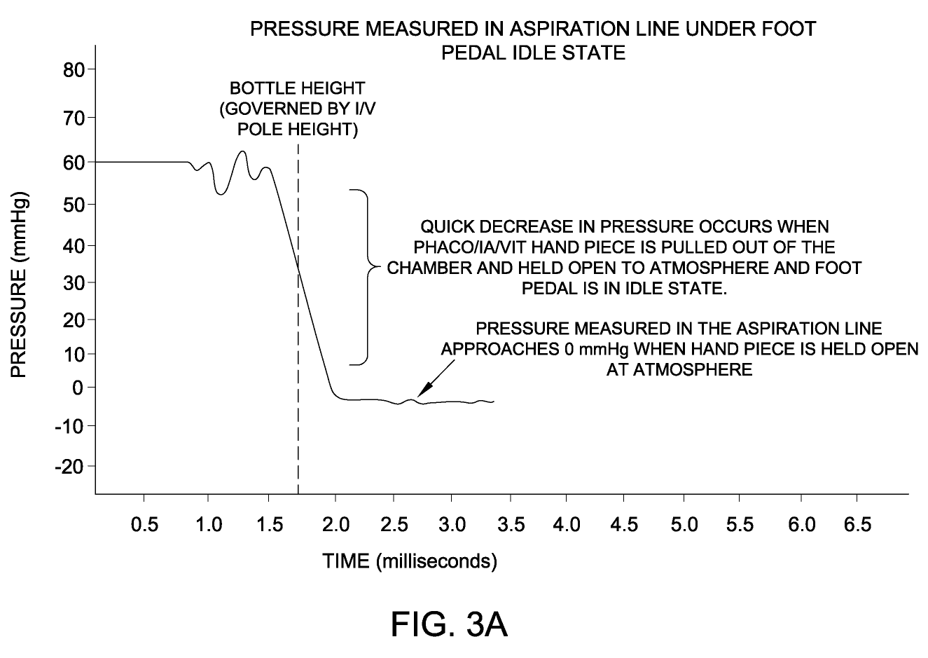

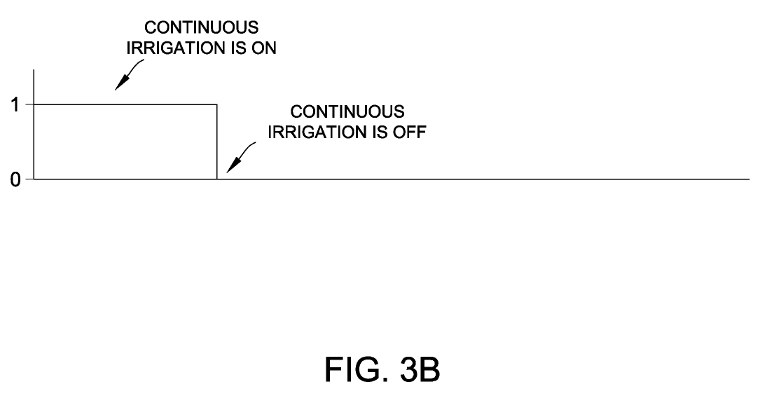

As illustrated in FIG. 3A, a rate of change of the aspiration pressure, as represented on the x-axis, versus time is shown when the Phaco/IA/Vit hand piece is pulled out of the eye chamber and held open to atmosphere. For example, aspiration pressure may be set to about 60 mmHg as at least partially influenced by gravity fed irrigation delivered to the surgical site. The removal of the surgical handpiece from the surgical site opens the handpiece to atmospheric pressure and the measured aspiration pressure may begin to rapidly drop towards 0 mmHg, creating a rate of change of less than about 60 mmHg/0.5 milliseconds. The auto-turn off feature of the present invention may use the aspiration line pressure measurements to determine if the hand piece is out of the surgical site and is under foot pedal idle state, which may then cause the continuous irrigation to be turned off. As illustrated in FIG. 3B, with its x-axis aligned with the FIG. 3A, the drop in aspiration pressure may trigger a continuous irrigation off condition. In an example of the present invention, a drop in pressure of more the 20 mmHg, for example, may be at least a partial trigger. Such a drop may, for example, occur in less than 5.0 milliseconds, at a rate of about 10 mmHg/millisecond, preferably in less than 4.0 milliseconds at a rate of about 10 mmHg/millisecond.

The triggers of various drops in pressure versus time may be associated with various predetermined thresholds and may encompass a wide range. For example, a predetermined drop in pressure may be in the range of about 15 mm Hgto about 40 mmHg and maybe preferably about 30 mmHg. The time over which a drop in pressure occurs may be from about 2.5 milliseconds to about 0.5 milliseconds. The shorter the time chosen may increase responsiveness of the system. Additionally, pairing these thresholds with other demonstrative features occurring within the use of the system, such as, for example, foot pedal position, aids in the elimination of switching off the continuous irrigation in a situation where it is still desired by the user of the system.

In alternate example, a GUI feature may be provided to select/deselect the continuous irrigation auto-on and auto-off feature in addition to continuous irrigation toggle button. In an example of the present invention, the continuous irrigation may be put into the off condition when the following trigger events are detected in phaco/IA/Vit mode:
1. Continuous irrigation is on, AND
2. Foot pedal treadle is at Idle (FP0) position, AND
3. Aspiration pressure is below an estimated IOP (e.g., the IOP may be determined by the governing bottle/bag height pressure minus the patient eye level), OR
4. Aspiration pressure has a rapid rate of change that is greater than an expected rate of change in pressure, e.g., the expected rate of change may be a typical rate of wound leakage or rate of change occurring under normal surgical procedures.

In an example of the present invention, the system may comprise a fluid source connected to an irrigation feed line, an aspiration line providing at least a partial aspiration vacuum at a surgical site, at least one pressure sensor in communication with the aspiration line, and at least one valve in communication with the irrigation feed line, wherein the at least one valve restricts fluid flow in the irrigation feed line in accordance with at least one measurement of aspiration pressure differential and at least one predetermined system attribute, and wherein the aspiration pressure differential is equal to a first measurement of aspiration pressure minus a second measurement of aspiration pressure over a predetermined time. The system may provide for a gravity fed fluid source and a pressurized fluid source. The aspiration pressure differential may be equal to about 20 mmHg and may be between about 20 mmHg and 40 mmHg. The predetermined time used by the system may range from about 2.5 milliseconds to about 0.5 milliseconds. Similarly, the system may make a first measurement upon a change in measured aspiration pressure of less than about 5%.

In an example of the present invention, a method for controlling fluid flow in a phacoemulsification surgical system is provided and comprises measuring a change in aspiration pressure, and restricting fluid flow in an irrigation feed line by at least one valve in accordance with the change in aspiration pressure and at least one system attribute, wherein the change in aspiration pressure may be equal to a first measurement of aspiration pressure minus a second measurement of aspiration pressure over a period of time. The at least one predetermined system attribute may be selected from the group consisting of foot pedal position, a minimum aspiration pressure; and a change in operation mode. The change in aspiration pressure may be equal to about 20 mmHg and may range between about 20 mm Hg and 40 mm Hg. The measuring may occur from about every 2.5 milliseconds to about every 0.5 milliseconds.

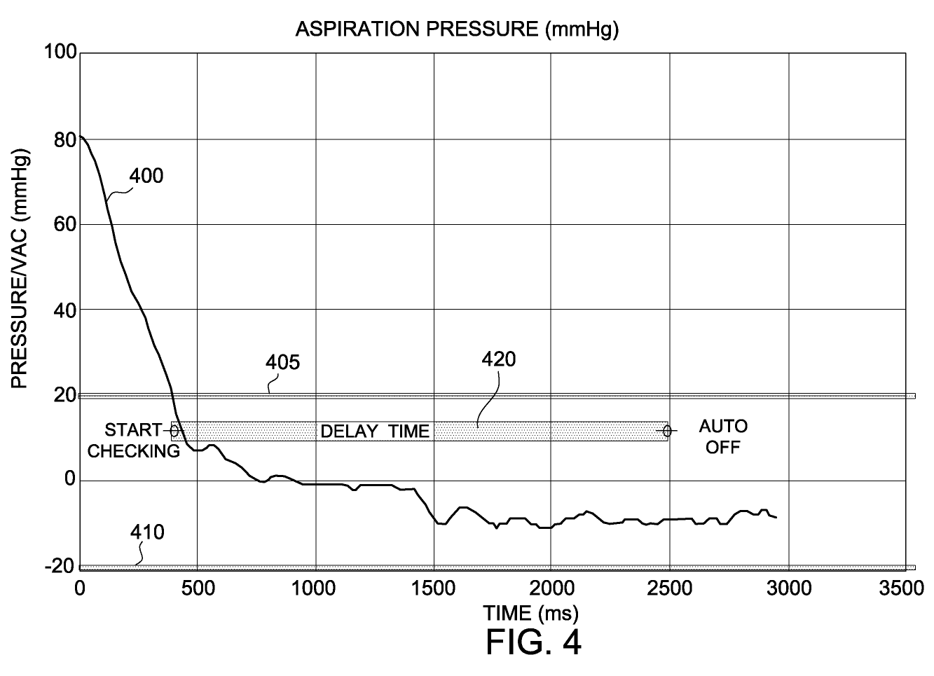

In an alternative example, the algorithm defines a band in which the aspiration vacuum 400 needs to fall in a specified time in order to automatically turn off the continuous irrigation as illustrated in FIG. 4. In this example, the algorithm may provide a user selection for delay time 420 with a band of pressure defined by upper pressure boundary 405 and lower pressure boundary 410. For example, an about 2000 millisecond time delay may be selected and may be programmed to be of any time delay desired by the user of the system. Once the following conditions are met, algorithm begins to increment a counter up to the user selected delay time. Once the user selected delay has elapsed, the algorithm may turn the continuous irrigation off.

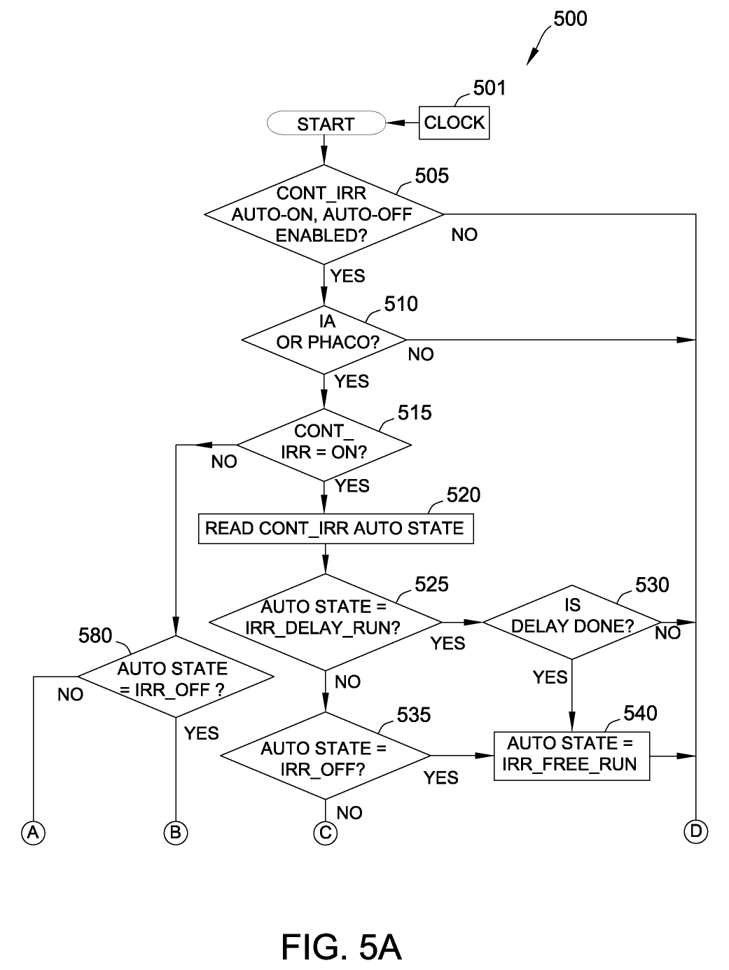

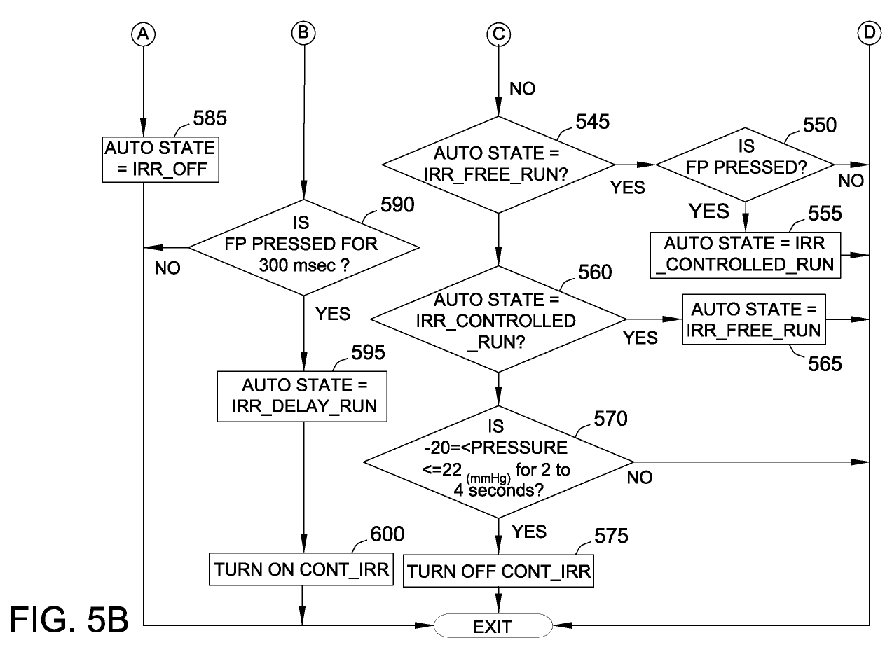

In an example illustrated in FIGS. 5A and 5B, certain conditions may trigger an example of the auto-off process 500 to begin a counter up to the user selected delay time utilizing clock 501 which may be associated with the system of the present invention. As illustrated, in step 505, the continuous irrigation auto-off feature may be enabled with the system checking to see if the system is in Phaco or IA surgical mode in step 510. If either mode is active, continuous irrigation may be turned on through step 515 which may lead into the continuous reading of irrigation state conditions, such as pressure and time, for example, at step 520. The continuous reading of irrigation state conditions may continue through other processes, such as the selected time delay at least partially administered through step 525 and step 530 such that a completed time delay may result in free running irrigation at step 540 and may lead to the exiting and repeating on the steps in FIGS. 5A and 5B. Alternatively, without a time delay and no automatic irrigation in step 535, free running irrigation may continue at step 545. If irrigation is in a controlled state at step 560, the irrigation may be returned to free running irrigation at step 565, otherwise the system will measure the aspiration pressure of the system at step 570 and will either turn off the continuous irrigation at step 575 or may return to the step 505. More specifically, step 570 may, for example, provide for the monitoring of aspiration pressure which may fall within pre-determined bands, such as between -20 mmHg to +22 mm Hg, and which may remain in this band for user selected delay. In an example, the pre-determined bands may have a lower limit as low as -100 mm Hg. Satisfaction of the step may turn off the continuous irrigation at step 575.

If back at step 515 continuous irrigation is not on, the system may check to see if the automatic irrigation state is not off at step 580. If the automatic irrigation state is not off, it may be turned off at step 585 before returning to the beginning of process 500. If the automatic irrigation state is off, the system may look to confirm whether a foot pedal associated with the system is actuated in a predetermined manner at step 590, which may result in setting the automatic state to an irrigation delay run at step 595 and may, in turn, activate continuous irrigation at step 600. Foot pedal actuation may include movement between zones, such as the foot pedal has traveled to at least Zone 1 and released to idle (Zone 0) and/or the foot pedal is depressed for a period of time.

Further, systems and methods for automatically turning irrigation off when the tip of the handpiece is out of the eye are disclosed. When the foot pedal is released, the system measures or estimates the pressure at the tip of the handpiece. If the pressure at the tip of the handpiece stays within a pressure range for a specified or predetermined period of time (delay time), the system determines the tip of the handpiece is outside of the eye and automatically turns off the continuous irrigation. The pressure at the tip of the handpiece can be measured or estimated using pressure readings from at least one pressure sensor. The pressure sensor may be located at or near the tip of the handpiece. Additionally, or alternatively, the at least one pressure sensor is coupled with the aspiration line and/or the irrigation line. The pressure at the tip of the handpiece may be measured using at least one pressure sensor coupled with the aspiration line. In another example, the pressure at the tip of the handpiece may be estimated based on measurements from at least one pressure sensor coupled with the irrigation line; and in another example, at least two pressure sensors coupled with the irrigation line may be used.

In another example, the pressure at the tip of the handpiece may be estimated using a pressure estimation algorithm and pressure readings from at least one pressure sensor coupled with the irrigation line. The pressure at the tip of the handpiece may be estimated using a pressure estimation algorithm, an irrigation pressure setpoint, and pressure readings from at least one pressure sensor located on the irrigation line by determining the fluidics resistance between the irrigation pressure setpoint and the at least one pressure sensor located on the irrigation line. In an example, the pressure measurement may be read and/or estimated every 0.5 milliseconds to about every 2.5 milliseconds

In an example, the phacoemulsification system includes an aspiration pump and at least one pressure sensor coupled with the aspiration line. The aspiration pump may be flow rate-based or pressure-based. When the foot pedal is released to idle (Zone 0) or is in Zone 1, there is typically no fluid flow in the aspiration line. When there is no fluid flow in the aspiration line and the tip of the handpiece is not inside the eye (e.g. the tip of the handpiece is in air), a pressure measured by at least one pressure sensor coupled with the aspiration line is typically about 0 mm Hg. However, when the foot pedal is released to idle (Zone 0), there are possible error contributions in the pressure reading. First, the height difference between the tip of the handpiece and the pressure sensor may result in a pressure differential because fluid pressure increases with depth due to the weight of the fluid above, as defined by Pascal’s law. Second, is pressure sensor inaccuracy. Stated another way, there may be some inaccuracy with the pressure sensor reading. Third, there is a possibility that instead of having no fluid flow when the foot pedal is released to idle (Zone 0), the aspiration line is exposed to the atmosphere for a pressure-based irrigation pump. In this case, there may be some pressure difference created when exposed to air. However, this pressure difference is likely minimal. Therefore, when there is no fluid flow in the aspiration line and the tip of the handpiece is not inside the eye (e.g. the tip of the handpiece is in air), the measured pressure is about atmospheric pressure (about 0 mmHg). For example, when there is no fluid flow in the aspiration line and the tip of the handpiece is not inside the eye (e.g. the tip of the handpiece is in air), the measured pressure is within a smaller pressure range. In an example, the smaller pressure range is about -10 mm Hg to about 10 mm Hg.

When there is no fluid flow in the aspiration line and the tip of the handpiece is inside the eye, the measured pressure is typically not within the smaller pressure range (e.g., a range of about -10 mm Hgto about 10 mmHg). Therefore, when the measured pressure is within the smaller pressure range continuous irrigation may be automatically turned off. In an example, when the measured pressure is within the smaller pressure range and at least one other condition is met, continuous irrigation is automatically turned off. For example, continuous irrigation is turned off if the measured pressure stays within the smaller pressure range for a delay time. The delay time may be programmed by a user and/or determined by at least one condition of the phacoemulsification system.

Additionally, or alternatively, the rate of change in aspiration pressure (i.e., aspiration vacuum pressure or aspiration vacuum) can also be used to determine whether the tip of the handpiece is outside of the eye. For example, when the rate of vacuum change is less than a predetermined threshold (e.g., 3 mm Hg/s /millisecond) continuous irrigation is automatically turned off. A predetermined threshold may be selected from the preferred range of about 2 mmHg/s to about 10 mm Hg/s.

In an example, if the measured pressure is within the smaller pressure range, a delay timer is activated (e.g., begins to increment a counter up to the delay time) and continuous irrigation is turned off only after expiration of the delay timer. The delay timer ensures the tip of the handpiece was deliberately taken out of the eye by the surgeon and eliminates or minimizes false positives. In an example, the delay time may be set from about 0.5 seconds to about 2 seconds and preferably set at about 0.5 seconds. The delay timer may be deactivated in response to one or more conditions. For example, the delay timer may be deactivated in response to the foot pedal deviating from an idle position (Zone 0), the measured pressure falling outside of the smaller pressure range, and/or a user manually deactivating the timer.

In an example, continuous irrigation is turned off if (i) the foot pedal is in Zone 0, (ii) vacuum pressure is within an aspiration vacuum pressure threshold range (e.g., about -10 mmHgto about 10 mmHg), and/or (iii) the rate of vacuum change is less than a predetermined threshold (e.g., 3 mmHg). In a further example, continuous irrigation is turned off if all of the foregoing conditions are met and a delay timer has expired (e.g., a delay time has elapsed).

In an example, the phacoemulsification system includes a flow rate-based irrigation pump and at least one pressure sensor coupled with the irrigation line. The flow rate-based irrigation pump may be any pump that commands a flow rate. Stated another way, the flow rate-based irrigation pump may be any pump in which a flow rate may be set. In examples in which the phacoemulsification system includes a flow rate-based irrigation pump, even when the foot pedal is released to idle (Zone 0), irrigation flow is non-zero due to the continuous irrigation. This flow rate creates a pressure differential between the tip of the handpiece and the location of the pressure sensor on the irrigation line. In an example, a pressure measurement is estimated using a pressure estimation algorithm, such as explained in U.S. Application No. 63/820,175, entitled “Systems And Methods To Detect Needles And Sleeves In A Phacoemulsification System” filed on Jun. 9, 2025, which hereby is incorporated by reference in its entirety.

When the tip of the handpiece is in the air, the estimated tip pressure is about atmospheric pressure (about 0 mmHg). However, when the foot pedal is released to idle (Zone 0), there are possible error contributions in the pressure reading. First, a flow rate reading inaccuracy could result in an inaccurate tip pressure estimation. Second, inaccuracy with fluidics resistance could result in an inaccurate tip pressure estimation. Third, the height difference between the tip of the handpiece and the pressure sensor may result in a pressure differential because fluid pressure increases with depth due to the weight of the fluid above, as defined by Pascal’s law. Fourth, is pressure sensor inaccuracy. Stated another way, there may be some inaccuracy with the pressure sensor reading.

When the system includes a flow rate-based irrigation pump and the tip of the handpiece is inside the eye, the estimated tip pressure is typically not within a smaller pressure range (e.g., about -10 mm Hg to about 10 mm Hg). Therefore, when the estimated tip pressure is within the smaller pressure range, continuous irrigation may be automatically turned off. Additionally or alternatively, the rate of change of the estimated tip pressure is used to determine whether the tip of the handpiece is outside of the eye. For example, if the rate of estimated tip pressure change is less than a predetermined threshold (e.g., 3 mm Hg), continuous irrigation may be automatically turned off. In an example, when the estimated tip pressure is within the smaller pressure range and at least one other condition is met, continuous irrigation is automatically turned off. For example, continuous irrigation is turned off if the estimated tip pressure stays within the smaller pressure range for a delay time. The delay time may be programmed by a user and/or determined by at least one condition of the phacoemulsification system, such as, for example, tip size and set intraocular pressure (IOP). Systems and methods for calculating estimated tip pressure and IOP is explained in U.S. Application No. 63/820,175, entitled “Systems And Methods To Detect Needles And Sleeves In A Phacoemulsification System” filed on Jun. 9, 2025, which hereby is incorporated by reference in its entirety.

In an example, if the estimated tip pressure is within the smaller pressure range, a delay timer is activated (e.g., begins to increment a counter up to the delay time) and continuous irrigation is turned off only after expiration of the delay timer. The delay timer helps to ensure the tip of the handpiece is in the air. In an example, the delay time may be about 0.5 second to about 2 seconds, and preferably about 0.5 seconds. The delay timer may be deactivated in response to one or more conditions. For example, the delay timer may be deactivated in response to the foot pedal deviating from an idle position (Zone 0), the measured pressure falling outside of the smaller pressure range, and/or a user manually deactivating the timer.

In an example, continuous irrigation is turned off if (i) the foot pedal is in Zone 0 or Zone 1, (ii) estimated tip pressure is within a smaller pressure range (e.g., about -10 mm Hgto about 10 mm Hg), and/or (iii) the rate of estimated IOP change is less than a predetermined threshold (e.g., 3 mm Hg). In a further example, continuous irrigation is turned off if all of the foregoing conditions are met and a delay timer has expired (e.g., a delay time has elapsed).

In an example, the system includes a pressure-based irrigation pump and at least one sensor coupled with the irrigation line. The pressure-based irrigation pump may be any pump that commands pressure. Stated another way, the pressure-based irrigation pump may be any pump in which an irrigation pressure may be set. In examples with the pressure rate-based irrigation pump, even when the foot pedal is released to idle (Zone 0), the set irrigation pressure is non-zero due to the continuous irrigation. This creates fluid flow from the irrigation pump to the tip of the handpiece when the tip has lower pressure than the set irrigation pressure. With a known fluid resistance, set irrigation pump pressure, and measured flow rate, tip pressure can be estimated. In an example, the pressure measurement at the tip of the handpiece is estimated using a pressure estimation algorithm. When the tip of the handpiece is in the air, the pressure at the tip of the handpiece is about atmospheric pressure (equivalent to about 0 mmHg gauge pressure). However, when the foot pedal is released to idle (Zone 0), there are possible error contributions to the pressure measurement. First, a flow rate reading inaccuracy could result in an inaccurate tip pressure estimation. Second, inaccuracy with fluidics resistance could result in an inaccurate tip pressure estimation. Third, the height difference between the tip of the handpiece and the pressure sensor may result in a pressure differential because fluid pressure increases with depth due to the weight of the fluid above, as defined by Pascal’s law. Fourth, is pressure sensor inaccuracy. Stated another way, there may be some inaccuracy with the pressure sensor reading.

When the system includes a pressure-based irrigation pump and the tip of the handpiece is inside the eye, the estimated tip pressure is typically not within the smaller pressure range (e.g., about -10 mm Hg to about 10 mm Hg). Therefore, when the estimated tip pressure is within the smaller pressure range, continuous irrigation may be automatically turned off. Additionally or alternatively, the rate of change of the estimated tip pressure is used to determine whether the tip of the handpiece is outside of the eye. For example, if the rate of estimated tip pressure change is less than a predetermined threshold (e.g., 3 mm Hg), continuous irrigation may be automatically turned off. In an example, when the estimated tip pressure is within the smaller pressure range and at least one other condition is met, continuous irrigation is automatically turned off. For example, continuous irrigation is turned off if the estimated tip pressure stays within the smaller pressure range for a delay time. The delay time may be programmed by a user and/or determined by at least one condition of the phacoemulsification system.

In an example, if the estimated tip pressure is within the smaller pressure range, a delay timer is activated (e.g., beings to increment a counter up to the delay time) and continuous irrigation is turned off only after expiration of the delay timer. The delay time helps to ensure the tip of the handpiece is in the air. In an example, the delay time may be about 0.5 seconds, about 1 second, or about 2 seconds. The delay timer may be deactivated in response to one or more conditions. For example, the delay timer may be deactivated in response to the foot pedal deviating from an idle position (Zone 0) or the foot pedal moving between positions (a pedal zone transition “PZT”), the measured pressure falling outside of the smaller pressure range, and/or a user manually deactivating the timer.

In an example, continuous irrigation is turned off if (i) the foot pedal is in Zone 0, (ii) estimated tip pressure is within a smaller pressure range (e.g., about -10 mm Hg to about 10 mmHg), and/or (iii) the rate of estimated IOP change is less than a predetermined threshold (e.g., 3 mm Hg). In a further example, continuous irrigation is turned off if all of the foregoing conditions are met and a delay timer has expired (e.g., a delay time has elapsed).

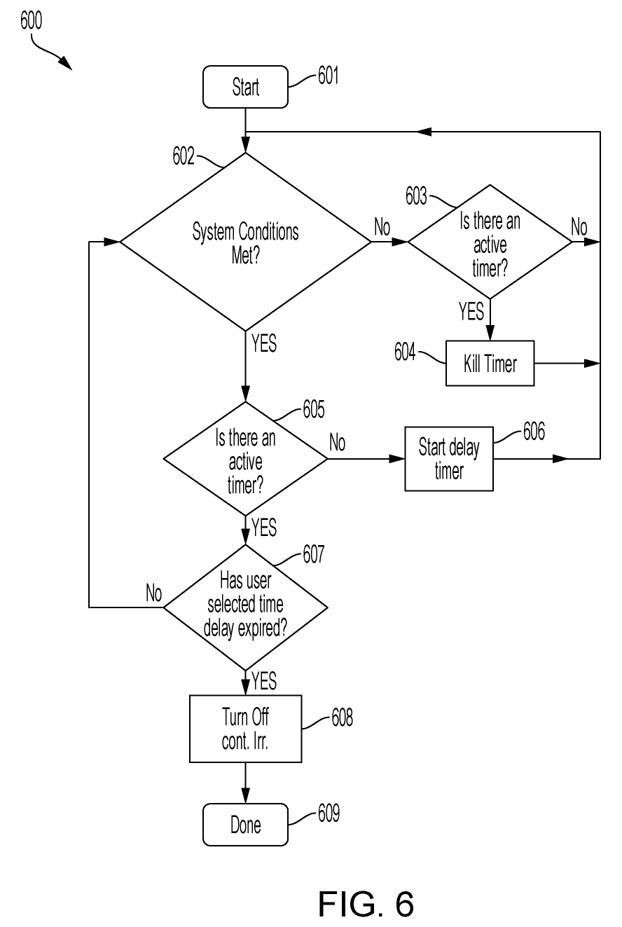

FIG. 6 is a flow diagram 600 for continuous irrigation of auto off (CIAO) At 601, the process starts.

At step 602, it is determined whether certain system conditions are met: Conditions at step 602 include the foot pedal position at Zone 0, but may otherwise vary between operational modes. As an example, the conditions for phaco mode may include a measured or estimated IOP between -20 mm Hg and 20 mm Hg and may further include an aspiration vacuum between -10 mm Hg and 20 mmHg. In another example, the conditions for I/A mode may include a measured or estimated IOP between -10 mmHgand 100 mmHg and may further include an aspiration vacuum between -10 mm Hg and 20 mm Hg. In yet another example, the conditions for vitrectomy mode may include a measured or estimated IOP between -25 mm Hg and 25 mm Hg and may further include an aspiration vacuum between -10 mm Hg and 20 mm Hg.

If neither of the conditions of step 602 are met, it is determined if there is an active delay timer at step 603. If there is an active delay timer, the delay timer is deactivated at 604, and the process returns to 602. If there is no active delay timer at step 603, the process also returns to step 602. If at least one of the conditions of step 602 are met, it is determined whether there is an active delay timer at 605. If it is determined that there is not an active delay timer, a delay timer is started at 606 and the process then returns to step 602. In an example, the delay time is about 0.5-2 seconds. In an example, the delay time is 0.5 seconds. In another example, the delay time is 1 second. In another example, the delay time is 2 seconds.

If it is determined that there is an active delay timer at 605, then it is determined whether the delay timer has expired (e.g., the delay timer has incremented up to the delay time) at step 607. If the delay timer has not expired, the process returns to step 602. If the delay timer has expired, continuous irrigation is turned off at step 608 and then the process ends at step 609.

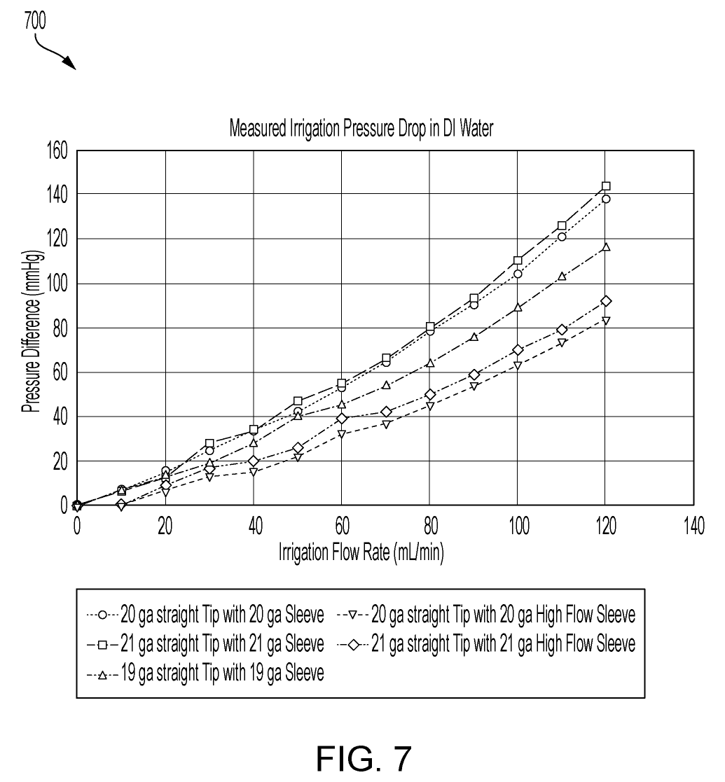

FIG. 7 is a graph 700 showing measured irrigation pressure drops (mmHg) of various handpiece tips, as represented on the y-axis, versus irrigation flow rate (mL/min), as represented on the x-axis. As illustrated in FIG. 7, the measured irrigation pressure difference increases as irrigation flow rate increases. For example, a handpiece having a 21 gauge straight tip with a 21 gauge sleeve (denoted by the square shaped symbol) measured a pressure difference of about 80 mmHg and a flow rate of about 80 ml/min. Similarly, a handpiece having a 21 gauge straight tip with a 21 gauge high flow sleeve (denoted by the diamond shaped symbol) measured a pressure difference of about 45 mmHg and a flow rate of about 80 ml/min. These two examples demonstrate the effect of sleeve type on pressure gradient. More generally, the data demonstrates a decrease of pressure difference of about 50%-70% percent based on whether or not a high flow sleeve is used.

Those of skill in the art will appreciate that the herein described apparatuses, engines, devices, systems and methods are susceptible to various modifications and alternative constructions. There is no intention to limit the scope of the invention to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual examples for the purpose of brevity in the disclosure. This method of disclosure is not to be interpreted as reflecting an intention that any subsequently claimed examples require more features than are expressly recited.

Further, the descriptions of the disclosure are provided to enable any person skilled in the art to make or use the disclosed examples. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein but rather is to be accorded the widest scope consistent with the principles and novel features disclosed herein.

1. A system for controlling fluid flow in an ophthalmic surgical system, the system comprising at least one processor in communication with at least one memory device, the at least one processor programmed to:
determine that a foot pedal is at zero position;
determine a pressure at the tip of a handpiece; and
disable a flow of irrigation fluid when the tip of the handpiece is removed from a surgical site;
wherein the at least one processor is configured to determine if the tip of the handpiece is removed from the surgical site at least based upon the determined pressure at the tip of the handpiece is within a predetermined pressure range after the expiration of a predetermined time delay.

2. The system of claim 1, wherein the predetermined pressure range for the system in phacoemulsification mode is between −20 mmHg and +20 mmHg.

3. The system of claim 1, wherein the predetermined pressure range for the system in irrigation/aspiration mode is between −10 mmHg and +10 mmHg.

4. The system of claim 1, wherein the predetermined pressure range for the system in vitrectomy mode is between −25 mmHg and +25 mmHg.

5. The system of claim 1, wherein the predetermined time delay is less than 0.5 seconds.

6. The system of claim 1, wherein the predetermined time delay is selected from a range of 0.5 seconds to 3.0 seconds.

7. The system of claim 1, wherein the at least one processor is further programmed to:
determine a system aspiration vacuum pressure; and
disable the flow of irrigation fluid only when the system aspiration vacuum pressure additionally falls within a predetermined aspiration vacuum range.

8. The system of claim 7, wherein the predetermined aspiration vacuum range is between −10 mmHgand +20 mmHg.

9. The system of claim 1, wherein the predetermined time delay is selected from a graphical user interface (GUI).

10. The system of claim 1, wherein the predetermined pressure range of the determined pressure at the tip of the handpiece is selected from a graphical user interface (GUI).

11. The system of claim 1, wherein the at least one processor is further configured to, in response to the foot pedal of the phacoemulsification surgical system no longer being in an idle position, disable the predetermined time delay.

12. A system for controlling fluid flow in an ophthalmic phacoemulsification surgical system, the system comprising at least one processor in communication with at least one memory device, the at least one processor programmed to:
receive an indication of a foot pedal position;
determine a pressure at the tip of a handpiece;
determine an operating mode of the system, the operating mode being one of a phacoemulsification mode, vitrectomy mode, and an irrigation/aspiration mode;
compare the determined pressure at the tip of the handpiece to a predetermined pressure range associated with the determined operating mode; and
disable a flow of irrigation fluid to a handpiece when the foot pedal position is at a zero position and the determined pressure at the tip of a handpiece falls within the predetermined pressure range associated with the determined operating mode for a predefined time period.

13. The system of claim 12, wherein the predetermined pressure range for the phacoemulsification mode is between −20 mmHg and +20 mmHg.

14. The system of claim 12, wherein the predetermined pressure range for the irrigation/aspiration mode is between −10 mmHg and +10 mmHg.

15. The system of claim 12, wherein the predetermined pressure range for the system in vitrectomy mode is between −25 mmHg and +25 mmHg.

16. The system of claim 12, wherein the at least one processor is further programmed to:
determine a system aspiration vacuum pressure; and
disable the flow of irrigation fluid only when the aspiration vacuum pressure falls within a predetermined aspiration vacuum range.

17. The system of claim 16, wherein the predetermined aspiration vacuum range is between −10 mmHg and +20 mmHg.

18. The system of claim 12, wherein the at least one processor is further configured to, in response to the foot pedal of the phacoemulsification surgical system no longer being in an idle position, reset the predefined time period.

19. The system of claim 12, wherein the predefined time period is defined by a user.

20. The system of claim 12, wherein the predefined time period is selected from a range of 0.5 seconds to 3.0 seconds.
